(19) United States
(12) Patent Application Publication
(10) Pub. No.: US 2009/0143678 A1
(43) Pub. Date: Jun. 4, 2009

(54) DEVICES FOR CREATING PASSAGES AND SENSING BLOOD VESSELS

(75) Inventors: Thomas M. KEAST, Sunnyvale, CA (US); Dave HAUGAARD, San Jose, CA (US); Edmund J. ROSCHAK, Mission Viejo, CA (US); Yaniv ROCK, Mountain View, CA (US)

Correspondence Address: LEVINE BAGADE HAN LLP, 2483 EAST BAYSHORE ROAD, SUITE 100, PALO ALTO, CA, 94303, US

(73) Assignee: BRONCUS TECHNOLOGIES, INC., MOUNTAIN VIEW, CA (US)

(21) Appl. No.: 12/323,198

(22) Filed: Nov. 25, 2008

Related U.S. Application Data
(63) Continuation of application No. PCT/US2007/070442, filed on Jun. 5, 2007.
(60) Provisional application No. 60/803,959, filed on Jun. 5, 2006.

(51) Int. Cl. A61B 8/12

(52) U.S. Cl. 600/439

(57) ABSTRACT

Devices and methods are disclosed for creating passages in tissue and detecting blood vessels in and around the passages. The devices may be used to create channels for altering gaseous flow within a lung to improve the expiration cycle of an individual, particularly individuals having Chronic Obstructive Pulmonary Disease (COPD). In addition, the devices may be used to sample tissue during biopsy or other medical procedures where perforating a blood vessel could result in injury to a patient.

### FIELD OF THE INVENTION

The invention is directed to devices for creating passages in tissue and detecting blood vessels in and around the passages. The device may be used to create channels for altering gaseous flow within a lung to improve the expiration cycle of an individual, particularly individuals having Chronic Obstructive Pulmonary Disease (COPD).

### BACKGROUND OF THE INVENTION

The American Lung Association (ALA) estimates that nearly 16 million Americans suffer from chronic obstructive pulmonary disease (COPD) which includes diseases such as chronic bronchitis, emphysema, and some types of asthma. The ALA estimated that COPD was the fourth-ranking cause of death in the U.S. The ALA estimates that about 14 million and 2 million Americans suffer from emphysema and chronic bronchitis respectively.

Those inflicted with COPD face disabilities due to the limited pulmonary functions. Usually, individuals afflicted by COPD also face loss in muscle strength and an inability to perform common daily activities. Often, those patients desiring treatment for COPD seek a physician at a point where the disease is advanced. Since the damage to the lungs is irreversible, there is little hope of recovery. Most times, the physician cannot reverse the effects of the disease but can only offer treatment and advice to halt the progression of the disease.

Lung volume reduction surgery is a procedure which removes portions of the lung that are over-inflated. The portion of the lung that remains has relatively better elastic recoil, providing reduced airway obstruction. The reduced lung volume also improves the efficiency of the respiratory muscles. However, lung reduction surgery is an extremely traumatic procedure which involves opening the chest and thoracic cavity to remove a portion of the lung. As such, the procedure involves an extended recovery period. Hence, the long term benefits of this surgery are still being evaluated. In any case, it is thought that lung reduction surgery is sought in those cases of emphysema where only a portion of the lung is emphysematous as opposed to the case where the entire lung is emphysematous. In cases where the lung is only partially emphysematous, removal of a portion of emphysematous lung which was compressing healthier portions of the lung allows the healthier portions to expand, increasing the overall efficiency of the lung. If the entire lung is emphysematous, however, removal of a portion of the lung removes gas exchanging alveolar surfaces, reducing the overall efficiency of the lung. Lung volume reduction surgery is thus not a practical solution for treatment of emphysema where the entire lung is diseased.

Both bronchodilator drugs and lung reduction surgery fail to capitalize on the increased collateral ventilation taking place in the diseased lung. There remains a need for a medical procedure that can alleviate some of the problems caused by COPD. There is also a need for a medical procedure that alleviates some of the problems caused by COPD irrespective of whether a portion of the lung, or the entire lung is emphysematous. The production and maintenance of collateral openings through an airway wall allows air to pass directly out of the lung tissue responsible for gas exchange. These collateral openings serve to decompress hyper inflated lungs and/or facilitate an exchange of oxygen into the blood.

It was found that creation of collateral channels in COPD patients allowed expired air to pass out of the lungs and decompressed hyper-inflated lungs. Such methods and devices for creating and maintaining collateral channels are discussed in U.S. Pat. No. 6,692,494; U.S. patent application Ser. Nos. 09/947,144, 09/946,706, and 09/947,126 all filed on Sep. 4, 2001; U.S. patent application Ser. No. 10/235,240 filed on Sep. 4, 2002; each of which is incorporated by reference herein in its entirety.

The creation of these channels also seems to overcome the shortcomings associated with bronchodilator drugs and lung volume reduction surgery. Placement of an implant within the channel, further increased the duration of the treatment.

However, because creation of the opening/channel is typically performed within the airway under bronchoscopic observation, care must be taken so as not to rupture a pulmonary vessel that lies beneath or outside of the airway wall. The need to avoid rupturing vessels that may be hidden by the airway walls is also evident when a surgeon attempts obtains a biopsy sample from within the bronchial tree. In addition, because the pattern of the pulmonary vessels varies between patients, care must also be taken when working within the channel or biopsy site. For instance, although a channel may be created without puncturing a blood vessel, the subsequent dilation, insertion of an implant, and/or removal of biopsy material may perforate vessels that were otherwise undetected during the creation of the channel.

In view of the above, a need remains to increase the safety when creating openings in tissue so as not to rupture a blood vessel.

### SUMMARY OF THE INVENTION

The invention relates to creation of passages while allowing sensing of blood vessels that may be in or around the area of the passage. Although specific reference is made to use of the subject invention within the lungs, it is noted that the invention may also be used within various other parts of the body that have a need for such safety measures.

The device allows for creating passages in tissue and sensing blood vessels in or around the passages. The device includes an elongate member having a near end and a far end, the far end including an ultrasound transducer assembly that is adapted to also mechanically pierce tissue and create an opening in the tissue when a tip of the device (or transducer) assembly is inserted into tissue. For example, the tip may be comprised of a plurality of elongate sections having increasing diameters (whether a discontinuous stepped increase or a continuous tapered increase) to create and dilate the opening. Variations of such devices may also include devices having a flexible distal end and stiff shaft sections to allow piercing of the tissue upon the application of axial force. The devices may be constructed to be of sufficient flexibility to navigate the tortuous path of a delivery device (introduced into tortuous anatomy) without piercing the wall of the relatively delivery device (e.g., a working channel of a bronchoscope), while having sufficient rigidity to pierce soft tissue.

In an additional variation, the devices described herein may also include an expandable member, such as a balloon or other mechanical means. When used in the lungs, the expandable member may comprise a balloon. The balloon may be constructed out of a distensible (or elastic) material. Alternatively, the balloon may be constructed form a non-distensible material. Such a material may be desirable when attempting to dilate strong or tough tissue. The balloon may also include an additional transducer assembly that permits scanning of the tissue before, after, or during dilation of an opening in tissue.

The inventive device is configured to communicate with an analyzing device or control unit adapted to recognize the reflected signal or measure the Doppler shift between the signals. As mentioned herein, the source signal may be reflected by changes in density between tissue. In such a case, the reflected signal will have the same frequency as the transmitted signal. When the source signal is reflected from blood moving within a vessel, the reflected signal has a different frequency than that of the source signal. This Doppler effect permits determination of the presence or absence of a blood vessel within tissue. The device may include a user interface which allows the user to determine the presence or absence of a blood vessel at the target site. Typically, the user interface provides an audible confirmation signal. However, the confirmation signal may be manifested in a variety of ways (e.g., light, graphically via a monitor/computer, etc.)

Although depicted as being external to the device, it is contemplated that the analyzing device may alternatively be incorporated into the device. The transducer assembly of the invention is intended to include any transducer assembly that allows for the observation of Doppler Effect, e.g., ultrasound, light, sound etc.

The invention also includes a method of treating lung tissue, method comprising selecting an area in lung tissue, examining the area of the lung tissue for the presence or absence of blood vessels, creating an opening in lung tissue; and examining the opening in the lung tissue for the presence or absence of blood vessels.

Examining the opening in the lung tissue may comprises inserting an ultrasound device into the opening in lung tissue to further identify the presence or absence of blood vessels beneath the surface of the lung tissue.

Examination of the area of lung tissue for the presence of blood vessels may include examining the area at a surface of the lung tissue with the ultrasound device.

The opening may be expanded with a member such as a balloon. As noted, a non-distensible balloon may allow for greater pressurization during the expansion of tissue.

### BRIEF DESCRIPTION OF THE DRAWINGS

FIGS. 1A-1C illustrate various states of the natural airways and the blood-gas interface.

FIG. 1D illustrates a schematic of a lung demonstrating a principle of the effect of collateral channels placed therein.

FIGS. 2A-2D illustrates variations of the inventive device.

FIGS. 3A-3B illustrate additional variations of the inventive device having sections of varying diameters to aid in dilating the opening.

FIGS. 4A-4D additional configurations of the device.

FIG. 4E illustrates the device as having an additional transducer located within the expandable member.

FIGS. 5A-5C illustrate a non-exhaustive sample of variations of the transducer assembly.

FIGS. 6A-6D illustrates one example of use of the device. In the illustrated example, the device creates a collateral channel in the airway wall tissue.

### DETAILED DESCRIPTION OF THE INVENTION

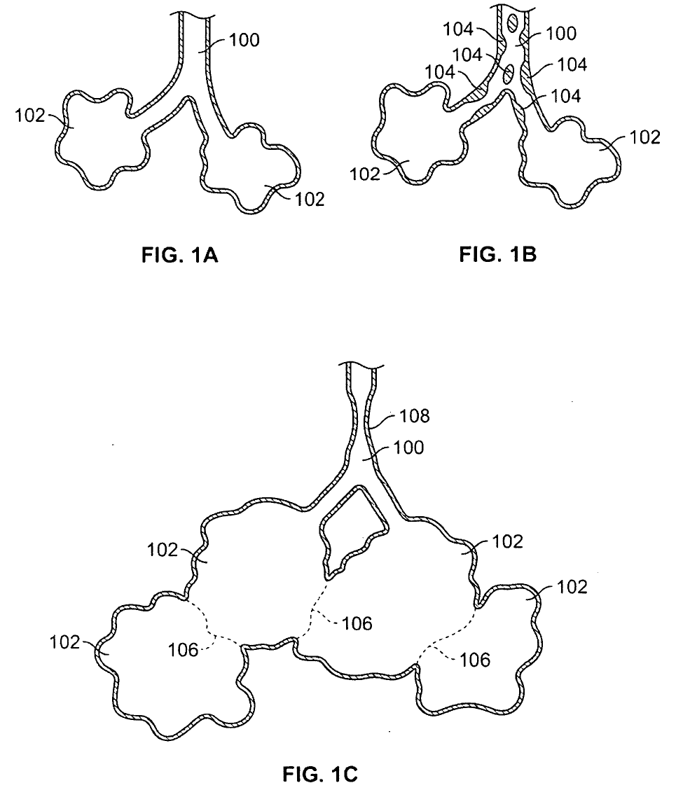

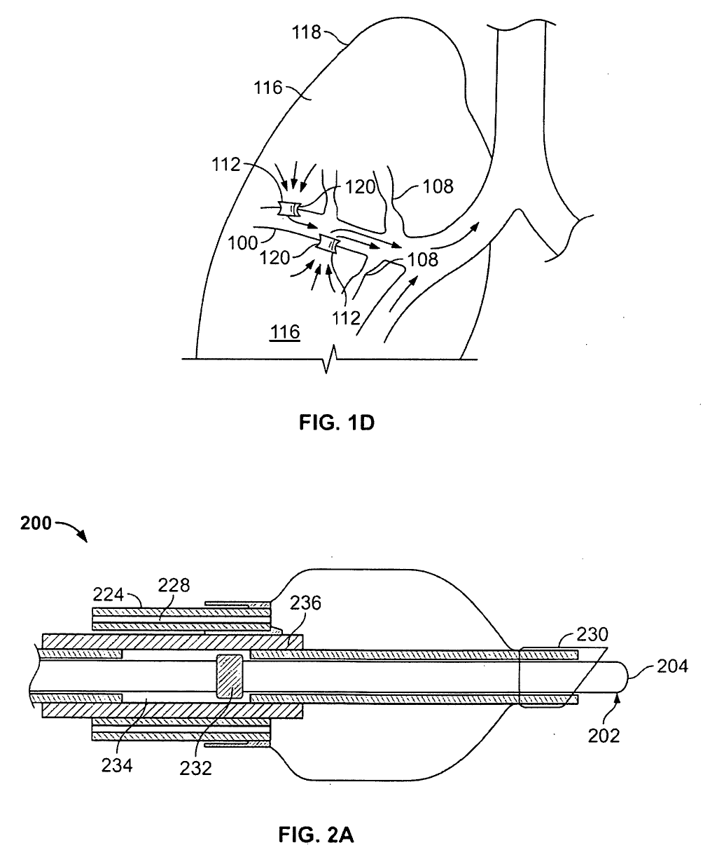

FIG. 1A shows a simplified illustration of a natural airway 100 which eventually branches to a blood gas interface 102. FIG. 1B illustrates an airway 100 and blood gas interface 102 in an individual having COPD. The obstructions 104 (e.g., excessive mucus resulting from COPD, sec above) impair the passage of gas between the airways 100 and the interface 102. FIG. 1C illustrates a portion of an emphysematous lung where the blood gas interface 102 expands due to the loss of the interface walls 106 which have deteriorated due to a bio-chemical breakdown of the walls 106. Also depicted is a constriction 108 of the airway 100. It is generally understood that there is usually a combination of the phenomena depicted in FIGS. 1A-1C. More usually, the states of the lung depicted in FIGS. 1B and 1C are often found in the same lung.

As will be explained in greater detail below, the production and maintenance of collateral openings or channels through airway walls permits expired air to pass directly out of the lung tissue and into the airways to ultimately facilitate exchange of oxygen into the blood and/or decompress hyper inflated lungs. The term ‘lung tissue’ is intended to include the tissue involved with gas exchange, including but not limited to, gas exchange membranes, alveolar walls, parenchyma, airway walls and/or other such tissue. To accomplish the exchange of oxygen, the collateral channels allow fluid communication between an airway and lung tissue. Therefore, gaseous flow is improved within the lung by altering or redirecting the gaseous flow within the lung, or entirely within the lung.

FIG. 1D illustrates a schematic of a lung 118 to demonstrate a benefit of the production and maintenance of collateral openings or channels through airway walls. As shown, a collateral channel 112 (located in an airway wall 110) places lung tissue 116 in fluid communication with airways 100 allowing expired air to directly pass out of the airways 100. The term channel is intended to include an opening, cut, slit, tear, puncture, or any other conceivable artificially created opening. As shown, constricted airways 108 may ordinarily prevent air from exiting the lung tissue 116. In the example illustrated in FIG. 1D, there is no implanted structure placed in the collateral channel 112. However, conduits or implants 120 may be placed in the collateral channels 112 to assist in maintaining the patency of the collateral channels 112. Examples of conduits may be found in the applications discussed above. While there is no limit to the number of collateral channels which may be created, it is preferable that 1 or 2 channels are placed per lobe of the lung. For example, the preferred number of channels is 2-12 channels per individual patient. In current trials, it was found that 1-4 channels placed per lobe of the lung and 4-16 channels per individual patient was preferable. This number may vary on a case by case basis. For instance, in some cases an emphysematous lung may require 3 or more collateral channels in one or more lobes of the lung.

The present invention includes the use of a device which is able to detect the presence or absence of a blood vessel by placing a front portion of the device in contact with tissue. One variation of the invention includes the use of Doppler ultrasound to detect the presence of blood vessels within tissue. However, the frequency of the signals is not limited to the ultrasonic range, for example the frequency may be within the range of human hearing, etc.

The ultrasound Doppler operates at any frequency in the ultrasound range but preferably between 2 Mhz-30 Mhz. It is generally known that higher frequencies provide better resolution while lower frequencies offer better penetration of tissue. In the present invention, because location of blood vessels docs not require actual imaging, there may be a balance obtained between the need for resolution and for penetration of tissue. Accordingly, an intermediate frequency may be used (e.g., around 8 Mhz). A variation of the invention may include inserting a fluid or gel into the airway to provide a medium for the Doppler sensors to couple to the wall of the airway to detect blood vessels. In those cases where fluid is not inserted, the device may use mucus found within the airway to directly couple the sensor to the wall of the airway.

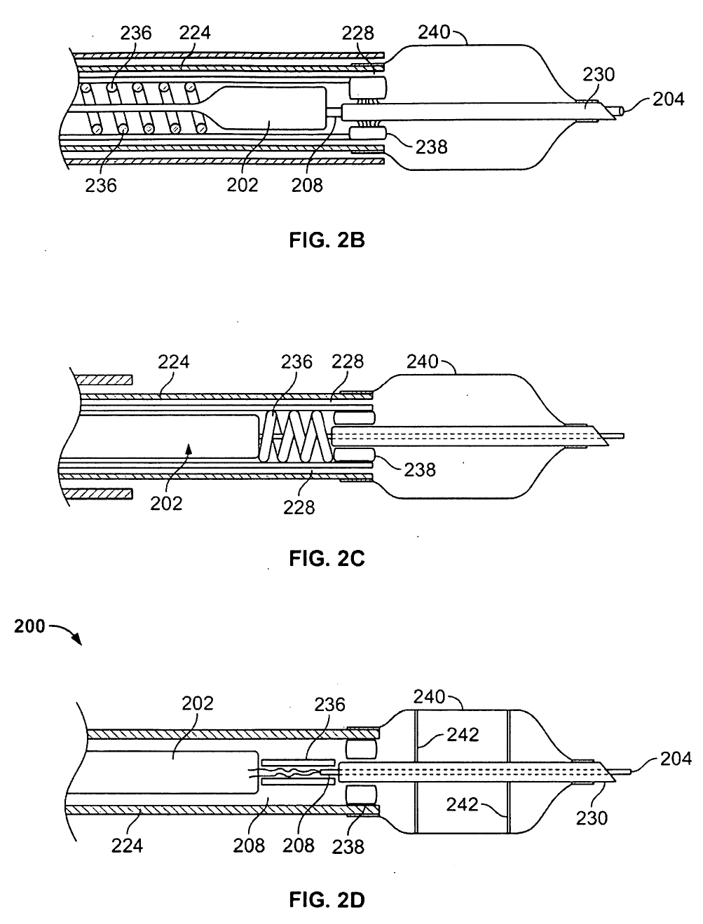

FIGS. 2A through 2D illustrate variations of devices 200 where the transducer assembly is located at a distal end of the device but is retractable within a needle tip of the device.

FIG. 2A illustrates a sectional side view of a variation of the inventive device 200. The device 200 includes a transducer assembly 202 having a tip 204 that is adapted to pierce tissue. Variations of the device may further include a sharpened tip or needle tip 230 in the event that the transducer tip alone is insufficient to pierce the tissue. It is contemplated that, throughout this disclosure, the transducer assembly 202 may be a transducer or a transducer coupled with a covering and other components (examples of which are discussed below). The transducer assembly of any variation of the present invention may be located within the elongate member, or it may be located within a portion of the tip 204 of the device.

In any case, the transducer assembly may or may not be configured to move relative to the tissue needle tip. The elongate member described herein may be comprised of any commercially available medical-grade flexible tubing. For example, the elongate member may comprise a PTFE material.

As shown in the variation illustrated in FIG. 2A, the transducer assembly 202 is able to advance out of or retract within the needle tip 230 as a limiter or hub 232 is affixed to the transducer assembly 202 to allow limited movement of the transducer assembly 202 within the device 200. When advanced through an access device or bronchoscope, the transducer tip 204 is located distally to the needle tip 230 to minimize the chance that the needle tip 230 damages the interior of the access device. The hub 232 is able to travel within a range 234 in the elongate member 224 to allow the transducer assembly 202 to be withdrawn into the needle assembly 230. Accordingly, a limiter assembly 236 can be placed within the elongate member 224 to control movement of the transducer assembly 230.

The transducer assembly may be coupled to a power supply in any standard manner. For example, the device may include a first conducting member and a second conducting member (e.g., wires) both extending through at least a portion of elongate member to the transducer assembly. The conducting members may extend through the lumen of the elongate member or may extend in the wall of the elongate member. In any case, the conducting members provide the energy and controls for the transducer assembly. For example, the conducting members may be coupled to an ultrasound source. Moreover, variations of the inventive device include conducting members which may be comprised of a series of wires, with one set of wires being coupled to respective poles of the transducer, and any number of additional sets of wires extending through the device. Ultimately, the wires enable the device to couple to energy and control units. Although not illustrated, any variation of the device may include an outer sheath in which the device may be advanced to a target tissue site.

The variation of the device depicted in FIG. 2A includes a needle tip 230 (e.g., a stainless steel thin walled tubing such as a hypo-rube, cannula tubing such as that used for needles, etc.) The sharp tip described herein will be sharp or have a sufficiently small surface area such that insertion of the tip through tissue may be performed by advancement of the device (or a component thereof). It is contemplated that, where possible, any of the tissue piercing members described herein may be incorporated into any of the variations described herein.

FIG. 2A also shows the device 200 as having an expandable member or a balloon member 240 that serves to dilate the opening created by the device. As shown, the balloon member 240 may be affixed to within the needle tip 230. This configuration allows for little or no transition as the balloon 240 is advanced within tissue. Once within tissue, the balloon is expanded to dilate tissue and allow for retraction of the device. The balloon 240 is inflated via one or more lumens 228 of the elongate member 224.

It is noted that variations of devices described herein may be constructed to be stiff and inflexible or can be designed to have sufficient flexibility, column strength and length to access the tissue targeted for treatment within tortuous anatomy (such as those devices intended for use in small airways of the lung). Accordingly, for devices used to create collateral channels within lungs, the length of the device should preferably be between 1.5-3 ft long in order to reach the targeted airways.

FIG. 2B illustrates a variation of a device 200 having an expandable member 240 affixed to a needle tip 230 where the transducer is located within the needle tip. Again, in such variations it may be desirable to minimize the transition between the needle tip 230 and the balloon to allow case of insertion of the uninflated balloon into the tissue opening. Accordingly, in this variation, the distal end of the expandable member 240 is affixed to the exterior of the needle and directly adjacent to the tip 230.

FIG. 2B also illustrates a spring member 236 coupled to the transducer assembly 202 where the spring 236 allows the transducer 208 and its tip 204 to withdraw into the needle given a certain amount force applied on the transducer tip 204 by the tissue. For example, if the tip 204 of the transducer assembly is unable to pierce tissue, once the tissue exerts a force beyond the threshold force on the transducer tip, the transducer assembly compresses the spring to retract the transducer tip. Naturally, the spring advances the transducer assembly out of the needle after removal of the force at the tip of the transducer assembly. In the illustrated variation, the device 200 also includes a spacer 238 to couple the needle 230 to the transducer assembly and/or elongate member. However, variations of the device include sizing the components to eliminate the need for the spacer 238.

FIGS. 2C-2D illustrate additional variations of tissue piercing transducer probes according to the present invention where the distal tip of the transducer assembly 204 is spring loaded. As shown in FIG. 2C, the spring may be a conventional coil or helical spring. Alternatively, or in combination, the spring may be a resilient tube 236 as shown in FIG. 2D. FIG. 2D also illustrates a balloon 240 as having marker bands 242. The marker bands 242 assist in placement of the balloon within the tissue wall being dilated.

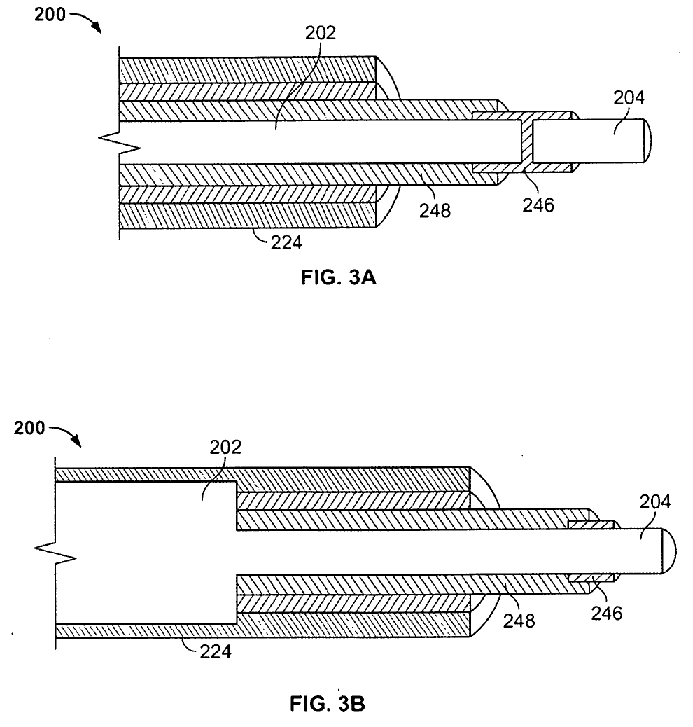

FIGS. 3A-3B show additional variations of devices 200 having transducer assemblies 202 configured to pierce tissue. As shown, these variations include fixed assemblies without needle tips (although modifications to include needle tips are within the scope of the invention). Instead, the transducer tip 204 is sufficient to puncture tissue when additional force is applied against the tissue. These variations show the distal end of the device 200 as having a series of dilation sections of increasing diameters, steps, or shoulders 246, 248. Instead of dilating the opening with a balloon member as shown herein, advancing the device 200 into tissue causes the dilation sections 246, 248 to dilate tissue. As shown, the dilation sections 246, 248 can include rounded transitions between the sections. Alternatively, but not shown, the dilation sections 246, 248 can have tapered transitions. Such configurations may allow for dilation of the opening to ease insertion of the expandable member within the opening.

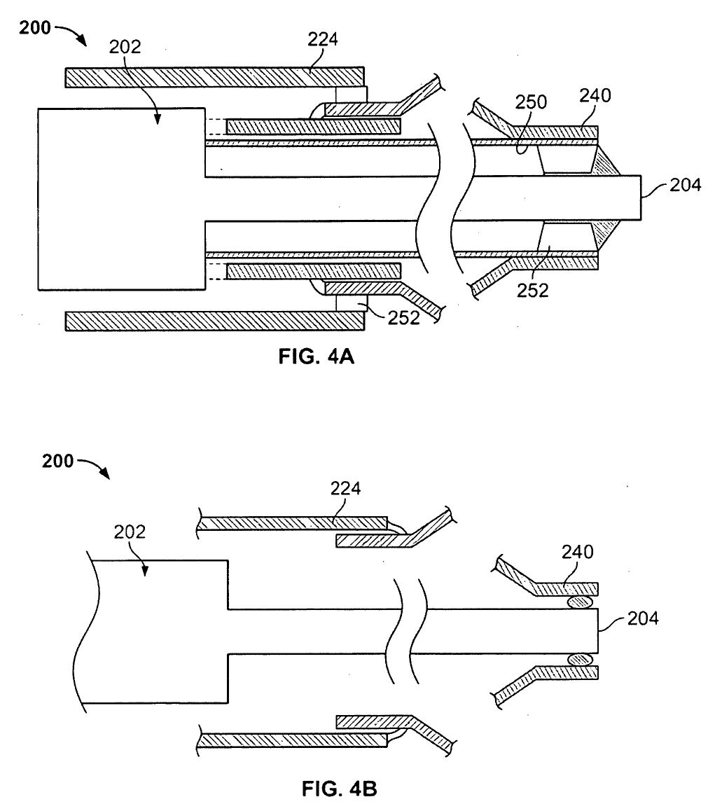

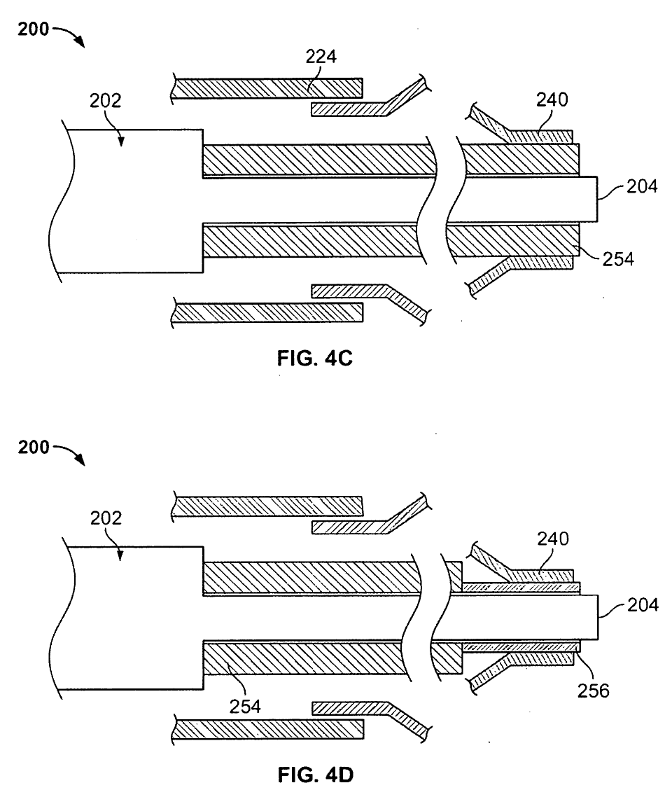

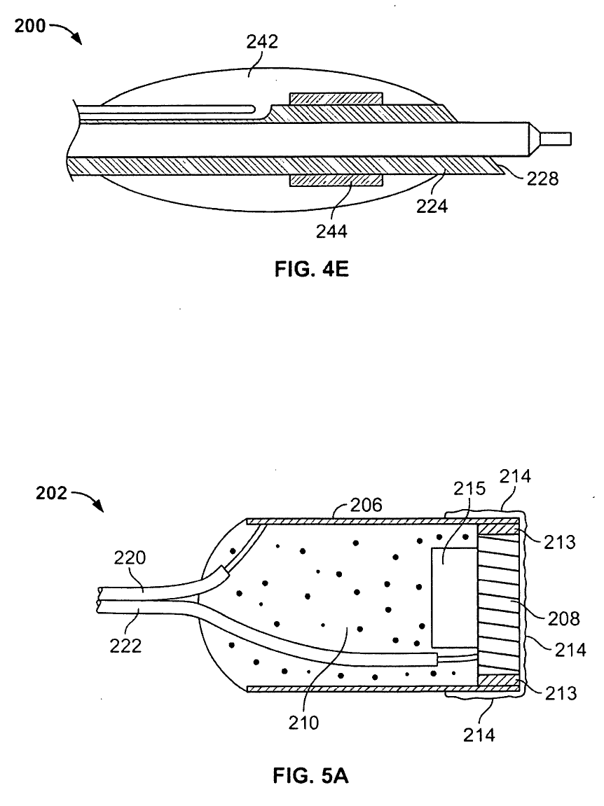

FIG. 4A-4D illustrates additional variations of the device with an expandable member or balloon 240. In FIGS. 4A-4D the balloon is partially shown to illustrate variations of affixing the balloon to the device to ease transition of the balloon into the tissue upon creation of an opening by a tip 204 of the device 200. The use of a balloon 242 allows dilation of the passage in tissue created by the transducer assembly lip. Variations of the invention can be designed for use in tough tissue that is resistant to radial expansion (such as an airway wall). In such variations, the balloon may comprise non-distensible balloon to overcome the toughness of the tissue. Non-distensible balloons are generally made up of relatively inelastic materials consisting of PET, nylons, polyurethanes, polyolefins, PVC, and other crosslinked polymers. Therefore, use of a non-distensible balloon allows for easier expansion of tissue because the non-distensible balloon permits high pressurization (> 6 atm). Moreover, non-distensible balloons generally inflate in a uniform shape since the balloon unfolds to assume an expanded shape. In contrast, distensible balloons typically expand in shape when pressurized. In any case, it should be noted that distensible and/or non-distensible balloons may be used in the present invention depending upon the application.

Non-distensible balloons typically occupy a greater mass than distensible balloons because the non-distensible balloon is inelastic and is folded in an unexpanded shape. Therefore, variations of the invention include non-distensible balloons having a working diameter (or diameter in an unexpanded shape) that is close to the diameter of the piercing member. This allows insertion of the unexpanded balloon into the opening created by the piercing member. Accordingly, balloons of the present invention may include thin walled balloons, balloons with small distal profiles, balloons with distal ends that are close in actual diameter to the diameter of the piercing member, or balloons that folds into low profile state, or balloons having a combination of these features.

FIG. 4A illustrates a variation of a device 200 having a balloon 240 having an end affixed to an elongate member 224 and a distal end affixed to a tip 204 of a transducer assembly 202. In this variation, an additional tube 250 can be used to stiffen the tip 204 to aid in insertion of the device 200 into tissue. The stiffening rube 250 and balloon 240 can be affixed to the tip 204 via a seal or adhesive 252, which, as shown can be tapered. One benefit of affixing the balloon 240 within the elongate member 224 is that an end of the elongate member forms a shoulder 252. This shoulder 252 can provide a stop or an area of increased resistance to allow proper placement of the balloon 240 within the tissue to be dilated.

FIG. 4B shows a variation of a device 200 similar to the variation shown in FIG. 4A. However, in this variation, the balloon 240 is affixed directly to an elongate member 224 and tip 204 of the transducer assembly 202. As noted above, this variation allows for a smooth transition as the balloon 240 follows the tip 204 of the device 200 as it penetrates tissue.

FIG. 4C illustrates a variation of a device 200 where a balloon 240 is affixed to a polymeric tube 254 or other support member that is bonded to a tip 204 of a transducer assembly 202. FIG. 4D shows another variation of a device 200 similar to that shown in FIG. 4C, where the distal end of the balloon 240 is bonded to a cannula or hypo tube 256. The cannula 256 is placed adjacent to a support tube 254. As discussed above, the cannula 256 can assist in penetration of the tissue.

FIG. 4E illustrates an additional variation of the device 200 that includes a second transducer 244 (such as a ring transducer) located within the balloon 240. In this variation, when the balloon expands to dilate a passage, the second transducer permits the balloon 240 to perform additional scans for blood vessels.

As discussed herein, for some variations of the invention it is desirable to minimize the size of the device especially at the distal end. Although the invention may be any size, it was found that an overall device diameter of 0.071″ was acceptable. In additional examples of the device, it was found that a tip ranging from 0.010″ to 0.025″ in diameter was acceptable to penetrate tissue.

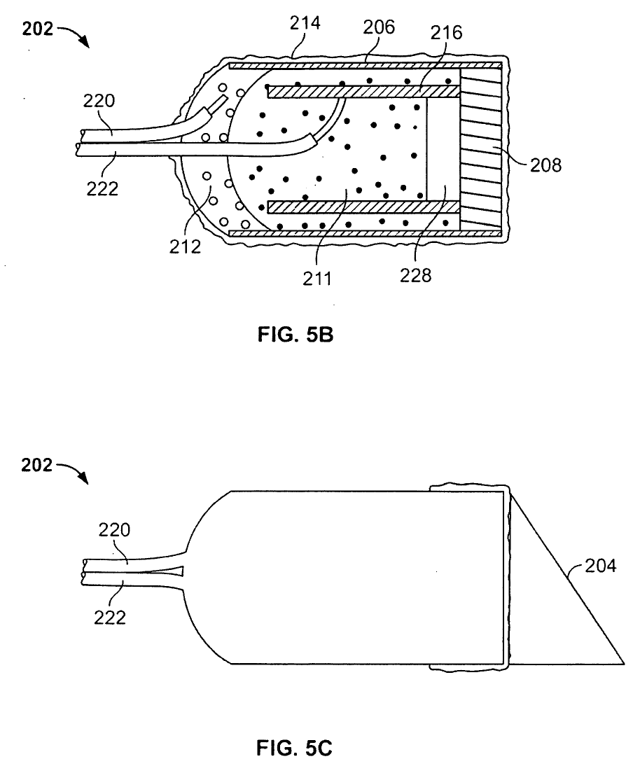

FIGS. 5A-5B illustrate a non-exhaustive sample of variations of the transducer assembly 202 configured to reduce an overall size of the assembly. It is noted that the invention may use any type of transducer assembly. FIG. 5A illustrates a cross-sectional view of a basic variation of a transducer assembly 202. The transducer assembly 202 includes at least one transducer 208 (e.g., a piezoelectric transducer.) In this variation, the front surface of the transducer 208 comprises a first pole and the rear surface comprises a second pole.

The transducer or transducers may comprise a piezo-ceramic crystal (e.g., a Motorola PZT 3203 HD ceramic). In the current invention, a single-crystal piezo (SCP) is preferred, but the invention docs not exclude the use of other types of ferroelectric material such as poly-crystalline ceramic piezos, polymer piezos, or polymer composites. The substrate, typically made from piezoelectric single crystals (SCP) or ceramics such as PZT, PLZT, PMN, PMN-PT; also, the crystal may be a multi layer composite of a ceramic piezoelectric material. Piezoelectric polymers such as PVDF may also be used. Micromachined transducers, such as those constructed on the surface of a silicon wafer are also contemplated (e.g., such as those provided by Sensant of San Leandro, Calif.) As described herein, the transducer or transducers used may be ceramic pieces coated with a conductive coating, such as gold. Other conductive coatings include sputtered metal, metals, or alloys, such as a member of the Platinum Group of the Periodic Table (Ru, Rh, Pd, Re, Os, Ir, and Pt) or gold. Titanium (Ti) is also especially suitable. The transducer may be further coated with a biocompatible layer such as Parylene or Parylene C.

The covering 206 of the transducer assembly 202 may contain at least a portion of the transducer 208. In some variations of the invention, the covering 206 may comprise a conductive material. In such cases the covering 206 itself becomes part of the electrical path to the first pole of the transducer 208. Use of a conductive covering 206 may require insulating material 213 between the sides of the transducer 208, thereby permitting a first conductive medium 214 to electrically couple only one pole of the transducer 208 to the covering 206.

At least a portion of the front surface of the transducer 208 will be in contact with the conductive medium 214. The conductive medium 214 permits one of the poles of the transducer 208 to be placed in communication with a conducting member that is ultimately coupled to a power supply. As shown in this example, the conductive medium 214 places the pole of the transducer 208 in electrical communication with the covering 206. In some variations the conductive medium 214 may coat the entire transducer 208 and covering 206. Alternatively, the conductive medium 214 may be placed over an area small enough to allow for an electrical path between a conducting member and the respective pole of the transducer 208. The conductive medium 214 may be any conductive material (e.g., gold, silver, tantalum, copper, chrome, or any bio-compatible conductive material, etc. The material may be coated, deposited, plated, painted, wound, wrapped (e.g., a conductive foil), etc. onto the transducer assembly 202.

The transducer assembly 202 depicted in FIG. 5A also illustrates conducting members 220, 222 electrically coupled to respective poles of the transducer 208. Optionally, the conducting members 220, 222 may be encapsulated within an epoxy 211 located within the covering 206. The epoxy 211 may extend to the transducer 208 thereby assisting in retaining both the conducting members 220, 222 and transducer 208 within the covering. It may also be desirable to maintain a gap 228 between the transducer 208 and any other structure. It is believed that this gap 228 improves the ability of the transducer assembly 202 to generate a signal.

FIG. 5B illustrates another variation of a transducer assembly 202. In this variation, the conductive medium 214 extends over the entire transducer covering 206. Accordingly, the covering 206 may be made of a non-conducting material (e.g., a polyamide tube, polyetherimide, polycarbonate, etc.) The transducer assembly 202 may further comprise a second tube 216 within the covering 206. This second tube 216 may be a hypo-tube and may optionally be used to electrically couple one of the conducting members to a pole of the transducer 208. As shown, the covering 206 may contain a non-conductive epoxy 210 (e.g., Hysol 2039/3561 with Scotchlite glass microspheres B23/500) which secures both the conducting member and the second tube 216 within the covering 206. This construction may have the further effect of structurally securing the transducer 208 within the assembly 202. Again, a gap 228 may or may not be adjacent to the transducer to permit displacement of the transducer 208.

FIG. 5B also illustrates the assembly 202 as having a conductive epoxy 212 which encapsulates the alternate conducting member 220. An example of a conductive epoxy is Bisphenol epoxy resin with silver particulates to enable conductivity. The particulates may be from 70-90% of the resin composition. The resin may then be combined with a hardener (e.g. 100 parts resin per 6 parts hardener.) The conductive epoxy 212 is in electrical communication with the conductive medium 214 allowing for a conductive path from the conducting member 220 to the conductive medium 214. Accordingly, use of the conductive epoxy 212 secures the conducting member 220 to the assembly 202 while electrically coupling the conducting member 220 to the transducer via the conductive coating 214.

Although variations of the transducer assembly include a tip and housing, the invention may omit the transducer covering and other structures not necessary to generate a source signal and receive a reflected signal. Therefore, it is contemplated that the invention may simply have a transducer that is coupled to a controller.

FIG. 5C illustrates another variation of a transducer assembly 202 in which a distal tip of the transducer 204 is sharpened to assist in puncturing tissue. Naturally, the tip may comprise any structure that assists in piercing tissue. In addition, the distal tip may be fabricated from materials that disperse the signal from the transducer or it may be fabricated from a material that does not interfere with the signal. In use, the practitioner can press the sharp tip against the tissue allowing for the tip to become embedded (wholly or partially) at the site. Next, once the practitioner determines whether the site is acceptable or not, the practitioner drives the transducer assembly 202 into the tissue to create an opening.

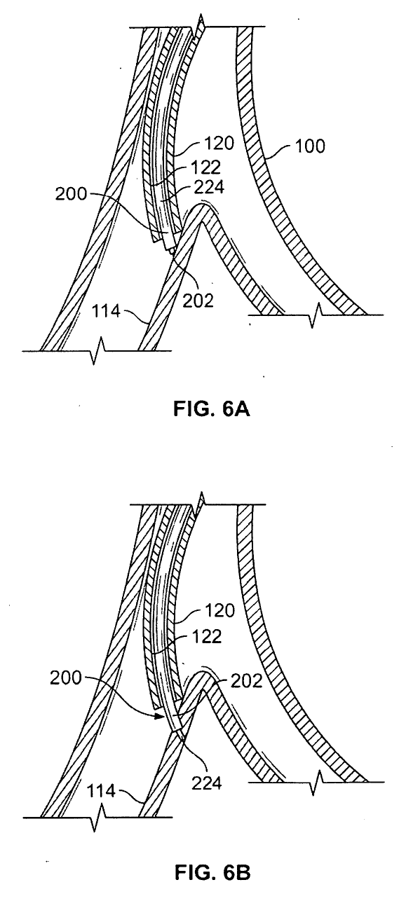

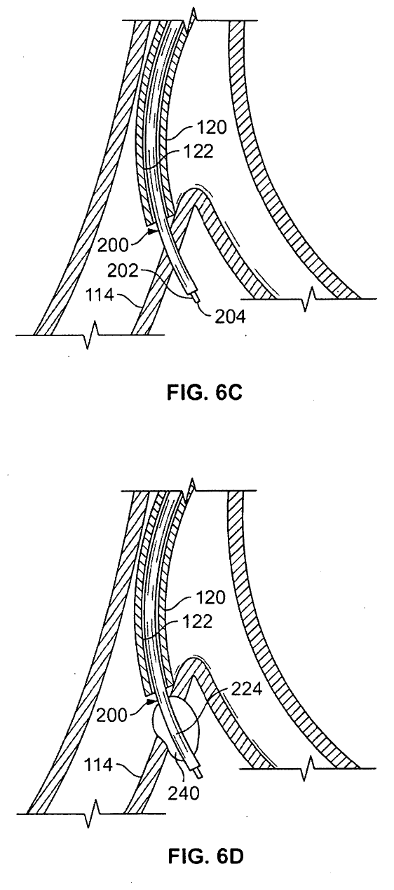

FIG. 6A-6C illustrates one example of use of the device. In the illustrated example, the device creates a collateral channel in the airway wall tissue within a lung. However, it is understood that the device may be used in any part of the body and for any application. For example, variations of the device may be used during a biopsy procedure to scan for blood vessels.

FIG. 6A illustrates optional use of an access device 120 advanced into the airways 100 of a lung. The access device may be a bronchoscope, endoscope, endotracheal tube with or without vision capability, or any type of delivery device. The access device 120 will have at least one lumen or working channel 122. The access device 120 will locate an approximate site 114 for creation of a collateral channel. For example, location of the site may be accomplished visually, or with additional equipment such as a CT scan to locate areas for treatment. In cases where the access device 120 is a bronchoscope or similar device, the access device 120 is equipped so that the surgeon may observe the site for creation of the collateral channel. In some cases it may be desirable for non-invasive imaging of the procedure. In such cases, the access device 120 as well as the other devices discussed herein, may be configured for detection by the particular non-invasive imaging technique such as fluoroscopy, “real-time” computed tomography scanning, or other technique being used.

FIG. 6A also illustrates advancement of a variation of the inventive device 200 through the channel 122 of the access device 120 towards the target site 114. The medical practitioner then uses the tip 204 of the transducer assembly 202 to inspect the target site to determine whether a blood vessel is adjacent to the site. If a blood vessel is detected, then another target site may be selected.

FIG. 6B illustrates the device 200 as the transducer assembly tip 204 acts as a tissue piercing member to creates a collateral channel (also referred to as an extra anatomic passage). In use, prior to creating the opening the device is pressed against tissue and deforms the tissue. At this point a medical practitioner can examine the tissue site with the device relatively still in view of the fact that it is deforming the tissue. Once the absence of a blood vessel is confirmed the practitioner further drives the ultrasound tip into tissue to create the opening. In addition, prior to creating the opening, the medical practitioner may scan other areas of the airway to affirmatively identify one or more blood vessels (or other structures) to ensure that the target site is free from blood vessels (or other structures).

It is noted that either the access device 120 or the inventive device 200 may be steerable. Such a feature may assist in the positioning of any of the devices used in the inventive method. Although it is not illustrated, as discussed herein, it is desirable to create the collateral channel such that it is in fluid communication with an air-sac. The fluid communication allows for the release of trapped gasses from the hyper-inflated lung.

FIG. 6C illustrates use of the device 200 to perform an additional scan for adjacent blood vessels. As shown, the device 200 can be inserted through the newly created passage to perform a scan for blood vessels underneath the surface of the tissue or within the opening of the passage. At any point, saline, other fluids or other substances may be inserted into and/or around the opening to assist in scanning the tissue.

FIG. 6D illustrates another step in which the opening is dilated by an expandable member 240. In this variation, the balloon 240 is located on the elongate member 224. As noted herein, in some variations of the invention, the balloon 240 is equipped with a second transducer. This configuration allows for additional scanning for blood vessels.

After dilation of the passage, the device may be removed. Alternatively, the expanded passage may be filled with fluid for additional scanning via the transducer assembly.

A further variation of the invention may include configuring the transducer assembly and/or controller to have different levels of sensitivity. For example, a first level of sensitivity may be used to scan the surface of tissue. Then, after creation of the opening, the second level of sensitivity may be triggered. Such a feature acknowledges that scanning of tissue on, for example, the airway wall may require a different sensitivity than when scanning tissue within the parenchyma of the lung.

It should be noted that the invention includes kits containing the inventive device with any one or more of the following components, a Doppler ultrasound controller, a conduit as described in one or more of the applications listed above, and a bronchoscope/endoscope.

In the above explanation of Figures, similar numerals may represent similar features for the different variations of the invention.

The invention herein is described by examples and a desired way of practicing the invention is described. However, the invention as claimed herein is not limited to that specific description in any manner. Equivalence to the description as hereinafter claimed is considered to be within the scope of protection of this patent.

The devices of the present invention are configured to locate a target site for creation of a collateral channel in the tissue and to create an opening in tissue. As discussed above, a benefit of this combination feature is that a single device is able to select a target location and then create an opening without having been moved. Although the device is discussed as being primarily used in the lungs, the device is not limited as such and it is contemplated that the invention has utility in other areas as well, specifically in applications in which blood vessels or other structures must be avoided while cutting or removing tissue (one such example is tumor removal).

The above illustrations are examples of the invention described herein. It is contemplated that combinations of aspects of specific embodiments/variations or combinations of the specific embodiments/variations themselves are within the scope of this disclosure.

1. A medical device for creating passages in tissue and sensing blood vessels in or around the passages, the device comprising:
an elongate member having a near end and a far end; and
an ultrasound transducer assembly adapted to pierce tissue to create an opening in the tissue when a tip of the ultrasound transducer assembly is inserted into the tissue; and
an expandable member coupled to the elongate member and adapted to dilate the opening.

2.-13. (canceled)

14. A medical device for creating passages in tissue and sensing blood vessels in or around the passages, the device comprising:
an elongate member having a near end and a far end; and
an ultrasound transducer assembly having a tip adapted to pierce tissue to create an opening in the tissue when a tip of the ultrasound transducer assembly is inserted into the tissue; and
a plurality of elongate dilation sections having increasing diameters greater than a diameter of the tip and less than a diameter of the elongate member, such that insertion of the elongate dilation sections dilates the opening in the tissue.

15.-21. (canceled)

22. A method of treating lung tissue, method comprising:
selecting an area in lung tissue;
examining the area of the lung tissue for the presence or absence of blood vessels with a device having an ultrasound transducer assembly;
creating an opening in the lung tissue with a tip of the ultrasound transducer assembly and advancing the lip of the ultrasound transducer assembly into the lung tissue; and
dilating the opening with the device.

23. The method of claim 22, where the tip of the ultrasonic transducer assembly is sharpened to penetrate tissue.

24. The method of claim 22, further comprising examining the opening in the lung tissue for the presence or absence of blood vessels.

25. The method of claim 24, where examining the opening in the lung tissue comprises inserting the tip of the ultrasound transducer assembly into the opening in lung tissue to further identify the presence or absence of blood vessels beneath the surface of the lung tissue.

26. The method of claim 24, where examining the area of lung tissue for the presence of blood vessels comprises examining the area at a surface of the lung tissue with the ultrasound device.

27. The method of claim 24, further comprising delivering a fluid to the opening.

28. The method of claim 27, where inserting the tip of the ultrasound transducer assembly into the opening in lung tissue occurs after delivering fluid to the opening.

29. The method of claim 22, where the device further includes an expandable member.

30. The method of claim 29, where the expandable member comprises a balloon.

31. The method of claim 30, where the balloon is a non-distensible balloon.

32. (canceled)

33. The method of claim 22, where a distal end of the device between the elongate member and the tip comprises a plurality of dilation sections of increasing size, such that dilating the opening with the device comprises advancing the dilation sections into the opening.

34. The method of claim 22, further comprising re-examining the dilated opening for the presence or absence of blood vessels beneath the surface of the lung tissue.

35. The method of claim 22, further comprising delivering an implant to the expanded opening.

36. The method of claim 22, where the tip of the ultrasound transducer assembly is spring loaded such that application of a sufficient force causes the tip to move into the device.

37. The method of claim 22, where creating the opening in the lung tissue with the tip of the ultrasound transducer assembly comprises first deforming lung tissue with the tip, then examining the area of the lung tissue for the presence or absence of blood vessels.

38. The method of claim 22, where the device further includes a needle tip located proximal to the tip of the ultrasound transducer.
